(19) United States
(12) Patent Application Publication
(10) Pub. No.: US 2008/0241112 A1
(43) Pub. Date: Oct. 2, 2008

(54) Therapy of Kidney Diseases and Multiorgan Failure with Mesenchymal Stem Cells and Mesenchymal Stem Cell Conditioned Media

(76) Inventor: Christof Westenfelder, Salt Lake City, UT (US)

Correspondence Address: BRINKS HOFER GILSON & LIONE, P.O. BOX 10395, CHICAGO, IL, 60610, US

(21) Appl. No.: 11/913,900

(22) PCT Filed: May 10, 2005
(86) PCT No.: PCT/US05/16489

§ 371 Date: Apr. 11, 2008

(51) Int. Cl. A61K 35/12; A61P 13/12; A61P 17/02; A61P 29/00

(52) U.S. Cl. 424/93.7

(57) ABSTRACT

Methods and a composition for the treatment of organ dysfunction, acute renal failure, multi-organ failure, early dysfunction of kidney transplant, graft rejection, chronic renal failure, wounds, and inflammatory disorders including media conditioned by mesenchymal stem cells are provided. Methods for modulation of growth factor and cytokine expression including administering a therapeutic amount of mesenchymal stem cells, endothelial cells derived from mesenchymal stem cells, or media conditioned by mesenchymal stem cells are also provided.

### FIELD OF THE INVENTION

The present invention generally relates to therapies for organ dysfunction, multi-organ failure, renal dysfunction, wound healing and inflammatory diseases. More particularly, the present invention relates to therapies using mesenchymal stem cells, endothelial cells derived from mesenchymal stem cells by predifferentiation, mesenchymal stem cell conditioned media or combinations thereof.

### BACKGROUND OF THE INVENTION

Multi-organ failure (MOF) remains a major unresolved medical problem. MOF develops in the most severely ill patients who have sepsis, particularly after major surgery or trauma. MOF is characterized by shock, acute renal failure (ARF), leaky cell membranes, dysfunction of lungs, liver, heart, blood vessels and other organs. Mortality due to MOF approaches 100% despite the utilization of the most aggressive forms of therapy, including intubation and ventilatory support, administration of vasopressors and antibiotics, steroids, hemodialysis and parenteral nutrition. In addition, healing of surgical or trauma wounds when infected, is seriously impaired, further contributing to recurrent infections, morbidity and death.

ARF is defined as an acute deterioration in renal function within hours or days, resulting in the accumulation of toxic metabolites that are normally eliminated by the kidney. The most common cause of ARF is ischemic injury of renal tubular and postglomerular vascular endothelial cells. The principal etiologies for this ischemic form of ARF include intravascular volume contraction, resulting from bleeding, thrombotic events, shock, sepsis, major cardiovascular surgery, arterial stenoses, and others. Nephrotoxic forms of ARF are caused by radiocontrast agents, and frequently used medications such as chemotherapeutic agents, antibiotics, cyclosporine and others. Patients most at risk for ARF include diabetics, patients having underlying kidney, vascular, liver and cardiac diseases, the elderly, patients having cancer and patients having low blood pressure from various causes.

Both ischemic and nephrotoxic forms of ARF result in death of tubular cells. Sublethally injured tubular cells dedifferentiate, lose their polarity and express vimentin, a mesenchymal cell marker, and Pax-2, a transcription factor that is normally only expressed in the process of mesenchymal-epithelial transdifferentiation in the embryonic kidney.

The kidney, even after severe acute insults, has the remarkable capacity of self-regeneration and consequent re-establishment of nearly normal function. Regeneration of injured nephron segments is thought to be the result of migration, proliferation and redifferentation of surviving tubular cells and parallel repair of endothelial cells. In severe ARF, the self-regeneration capacity of the surviving tubular and endothelial cells is exceeded. Patients with isolated ARF from any cause, i.e., ARF that occurs without MOF, continue to have a mortality in excess of 50%. This dismal prognosis has not improved despite intensive care support, hemodialysis, and the recent use of atrial natriuretic peptide, Insulin-like Growth Factor-I (IGF-I), more biocompatible dialysis membranes, continuous hemodialysis, and other interventions.

Another acute form of renal failure, transplant-associated acute renal failure (TA-ARF), often develops due to kidney transplantation. The kidney recipients regularly develop early graft dysfunction (EGD), resulting in loss of kidney function and requiring treatment with hemodialysis until graft function recovers. The risk of TA-ARF is increased with elderly and very young donors, marginal graft quality, and an extended period of time between harvest of the donor kidney from a cadaveric donor and its implantation into the recipient, known as “cold ischemia time”. Early graft dysfunction due to TA-ARF has serious long-term consequences, including accelerated graft loss due to progressive, irreversible loss in kidney function that is initiated by TA-ARF and an increased incidence of acute rejection episodes leading to premature loss of the kidney transplant. Therefore, a need exists to provide a treatment or prevention of early graft dysfunction due to TA-ARF.

Chronic renal failure (CRF) is the progressive loss of nephrons and subsequent loss of renal function. Glomerular, vascular and inflammatory injuries to the kidney collectively result in the eventual loss of nephrons and end stage renal disease. The final common pathway in essentially all forms of CRF is a self-perpetuating fibrotic and sclerosing process most prominently manifested in the renal interstitium.

Many renal as well as non-renal disorders are also characterized by a highly pro-inflammatory state. For example, infection and sepsis in patients with end stage renal disease lead to high mortality rates. Various growth factors and cytokines play a central role in the pathophysiology of these disorders. Modulating the levels of such growth factors and cytokines will provide a method of treating patients having disorders including an inflammatory response.

Taken together, therapies that are currently utilized in the prevention of ARF, the treatment of established ARF of native kidneys per se or as part of MOF, and ARF of the transplanted kidney, organ failure in general, and inflammation have not succeeded to significantly improve morbidity and mortality in this large group of patients. Consequently, there exists an urgent need for the improved treatment of MOF, renal dysfunction, organ failure, and inflammatory disorders.

The present invention provides mesenchymal stem cells, mesenchymal cell-derived endothelial cells, and conditioned media from mesenchymal stem cells for treating MOF, renal dysfunction, organ failure, and inflammatory and degenerative disorders and for modulating expression of growth factors and cytokines in the injured organs of these patients in these patients.

### BRIEF SUMMARY

In order to alleviate one or more shortcomings of the prior art, a methods of treatment and a composition are provided herein.

FIG. 1b is a graph of serum creatinine levels following MSC injection 24 hours after reflow. Compared to vehicle treated control animals with identical, moderate acute renal failure, the administration of MSC 24 hours after reflow shows significant improvement in renal function;

FIG. 2a is a graph of serum creatinine levels for cell injections immediately after reflow showing improvement in renal function in rats having severe ARF with administration of MSC, a beneficial effect that was not obtained in vehicle or fibroblast infused control animals;

FIG. 2b is a graph of injury scores showing improvement in injury score with MSC administration;

FIG. 2c is a graph of PCNA staining showing increased numbers of proliferating cells with MSC administration;

FIG. 2d is a graph of the apoptotic index showing decreased numbers of apoptotic cells with MSC administration;

FIG. 3a shows cytokine expression in whole kidney;

FIG. 3b shows growth factor expression in whole kidney;

FIG. 3c shows expression of apoptotic and NOS genes in whole kidney; and

FIG. 4 shows Dox regulatable Epo expression in MSC.

### DETAILED DESCRIPTION OF THE INVENTION

The present invention will utilize mesenchymal stem cells, mesenchymal stem cell-derived endothelial cells, conditioned media derived from mesenchymal stem cells, and combinations thereof for the repair of damaged tissues, amelioration and prevention of tissue and organ damage in patients at risk for tissue damage and for the modulation of cytokine and growth factor expression levels within the damaged organs. In one aspect of the present invention, mesenchymal stem cells (MSC) may be administered to a patient in need thereof. The administration of MSC may be used in the treatment or prevention of multi-organ failure; kidney dysfunction including, but not limited to acute renal failure of native kidneys, ARF of native kidneys in multi-organ failure, ARF in

In one aspect of the present invention, a method of treating organ dysfunction, acute renal failure, multi-organ failure, early dysfunction of kidney transplant, graft rejection, chronic renal failure, wounds, and inflammatory disorders is provided. The method includes delivering a therapeutic amount of a pharmaceutically acceptable media that has been conditioned by exposure to mesenchymal stem cells (MSC) to a patient in need thereof.

In another aspect of the present invention, a composition is provided. The composition comprises a pharmaceutically acceptable media that has been conditioned by exposure to MSC.

In another aspect of the present invention, a method of modulating expression of at least one growth factor in an injured organ of a patient is provided. The method includes administering an effective amount of MSC, EC or MSC-conditioned media to the patient to modulate expression of the growth factor.

In another aspect of the present invention, a method of modulating expression of at least one cytokine in an injured organ of a patient is provided. The method includes administering an effective amount of MSC, EC or MSC-conditioned media to the patient to modulate expression of the cytokine.

Advantages of the present invention will become more apparent to those skilled in the art from the following description of the preferred embodiments of the invention which have been shown and described by way of illustration. As will be realized, the invention is capable of other and different embodiments, and its details are capable of modification in various respects. Accordingly, the drawings and description are to be regarded as illustrative in nature and not as restrictive.

### BRIEF DESCRIPTION OF SEVERAL VIEWS OF THE DRAWINGS

FIG. 1a is a graph of serum creatinine levels for MSC injection immediately post reflow. Compared to vehicle treated control animals with identical, moderate acute renal failure, the administration of MSC directly after reflow shows significant improvement in renal function; transplanted kidneys; organ dysfunction; wound repair; and inflammatory disease. MSC may also be used to modulate expression of inflammatory cytokines and growth factors in patients, such as in the treatment or prevention of inflammatory diseases. MSC may be administered to treat or prevent additional disorders as will be understood by one of skill in the art.

In another aspect of the present invention, media conditioned by exposure to MSC in culture (MSC CM) may be administered to a patient in need thereof. By way of example, but not limited to the following, MSC CM may be used in the treatment or prevention of multi-organ failure, kidney dysfunction, including but not limited to, acute renal failure of native kidneys, ARF of native kidneys in multi-organ failure, ARF in transplanted kidneys, organ dysfunction, wound repair, and inflammation. MSC CM may also be used to modulate expression of inflammatory cytokines and growth factors in a patient, such as in the treatment or prevention of inflammatory diseases. MSC CM may be administered to treat or prevent additional disorders as will be understood by one of skill in the art.

### DEFINITIONS

The term “stem cell” refers to any cell that has the ability to self renew and to differentiate into a variety of cell types. The stem cells used herein are “adult” stem cells meaning that the stem cells are not embryonic in origin.

The term “culture” or “cell culture” refers to one or more cells within a defined boundary such that the cell(s) are allotted space and growth conditions typically compatible with cell growth or sustaining its viability. Likewise, the term “culture,” used as a verb, refers to the process of providing said space and growth conditions suitable for growth of a cell or sustaining its viability.

The term “conditioned media” refers to media that has been exposed to cells grown in culture for a time sufficient to include at least one additional component in the media, produced by the cells, that was not present in the starting media. The conditioned media for use in the present invention is removed from the cells in culture and filtered through a 0.22 μM filter to sterilize the conditioned media and to remove any cells, cell fragments and particulates. The starting media may be any media known to one of skill in the art, including commercially available media from vendors, for example, LifeTechnologies-GibcoBRL, Rockville, Md.; Sigma-Aldrich, Saint Louis, Mo.; and BioWhittaker, Walkersville, Md. Media used for administration to a patient in need thereof is prepared as a pharmaceutically acceptable composition, i.e. in a form appropriate for in vivo applications. Generally, this will entail preparing media compositions that are essentially free of pyrogens, as well as other impurities that could be harmful to humans or animals. Pharmaceutically acceptable media are commercially available from venders such as those listed above. The phrase “pharmaceutically acceptable” refers to molecular entities and compositions that do not produce adverse, allergic, or other untoward reactions when administered to an animal or a human. Supplementary active ingredients also can be incorporated into the compositions.

The term “therapeutically effective amount” refers to an amount of conditioned media or stem cells that is nontoxic but sufficient to provide the desired effect and performance at a reasonable benefit/risk ratio attending any medical treatment.

The term “therapeutically effective time” refers to the period of time during which a therapeutically effective amount of a conditioned media or stem cells is administered, and that is sufficient to reduce one or more symptoms of a condition.

The term “treating” refers to ameliorating at least one symptom of a condition.

The term “condition” is used to refer to a disease and/or a response to injury (e.g., trauma, etc.) or treatment (e.g., surgery, transplantation of tissue from a donor, etc.).

The term “growth factor” refers to a protein, a polypeptide, or a complex of polypeptides, including cytokines, that are produced by a cell and which can effect itself and/or a variety of other neighboring or distant cells. Typically growth factors affect the growth and/or differentiation of specific types of cells, either developmentally or in response to a multitude of physiological or environmental stimuli. Exemplary growth factors include, but are not limited to: insulin, insulin-like growth factor (IGF), nerve growth factor (NGF), Vascular Endothelial Growth Factor (VEGF), keratinocyte growth factor (KGF), fibroblast growth factors (FGFs), including basic FGF (bFGF), platelet-derived growth factors (PDGFs), including PDGF-AA and PDGF-AB, hepatocyte growth factor (HGF), transforming growth factor alpha (TGF-α), transforming growth factor beta (TGF-β), including TGF-β, and TGF-β₃, epidermal growth factor (EGF), granulocyte-macrophage colony-stimulating factor (GM-CSF), granulocyte colony-stimulating factor (G-CSF), growth hormone interleukins, prostaglandins, and the like.

The term “cytokine” or “cytokines” as used herein refers to the general class of biological molecules which have an effect on cell-cell interactions and that regulate the duration and intensity of an immune response. These molecules also regulate processes taking place in the extracellular environment.

The term “pro-inflammatory cytokines” include, but are not limited to, tumor necrosis factor-alpha (TNF-α), interleukin-beta (IL-1β), interferon-gamma (IFN-γ), interleukin-6 (IL-6), interleukin-8 (IL-8), lipopolysaccharide-binding protein, soluble lipopolysaccharide receptors (CD-14), and chemokines. A chemokine refers to a member of the superfamily of forty or more small (approximately about 6 to about 14 kDa) inducible and secreted pro-inflammatory polypeptides that act primarily as chemoattractants and activators of specific leukocyte cell subtypes.

The term “anti-inflammatory cytokines” include, but are not limited to, soluble TNF receptors (TNF-RI and TNF-RII), interleukin receptor antagonist (IL-1ra), interleukin-4 (IL-4), interleukin-10 (IL-10), interleukin-12 (IL-12), interleukin-13 (IL-13), and transforming growth factor-beta (TGF-β).

The term “inflammatory” when used in reference to a disease or condition refers to a pathological process caused by, resulting from, or resulting in inflammation that is inappropriate and/or does not resolve in the normal manner. Inflammation results in response to an injury or abnormal stimulation caused by a physical, chemical, or biologic agent; these reactions include the local reactions and resulting morphologic changes, destruction or removal of the injurious material, and responses that lead to repair and healing. Inflammatory disease and conditions may be systemic or localized to particular tissues or organs.

Inflammation is known to occur in many disorders which include, but are not limited to: Systemic Inflammatory Response (SIRS); Alzheimer's Disease (and associated conditions and symptoms including: chronic neuroinflammation, glial activation; increased microglia; neuritic plaque formation; and response to therapy); Amyotropic Lateral Sclerosis (ALS), arthritis (and associated conditions and symptoms including, but not limited to: acute joint inflammation, antigen-induced arthritis, arthritis associated with chronic lymphocytic thyroiditis, collagen-induced arthritis, juvenile arthritis; rheumatoid arthritis, osteoarthritis, prognosis and streptococcus-induced arthritis, spondyloarthopathies, gouty arthritis), asthma (and associated conditions and symptoms, including: bronchial asthma; chronic obstructive airway disease; chronic obstructive pulmonary disease, juvenile asthma and occupational asthma); cardiovascular diseases (and associated conditions and symptoms, including atherosclerosis; autoimmune myocarditis, chronic cardiac hypoxia, congestive heart failure, coronary artery disease, cardiomyopathy and cardiac cell dysfunction, including: aortic smooth muscle cell activation; cardiac cell apoptosis; and immunomodulation of cardiac cell function; diabetes and associated conditions and symptoms, including autoimmune diabetes, insulin-dependent (Type 1) diabetes, diabetic periodontitis, diabetic retinopathy, and diabetic nephropathy); gastrointestinal inflammations (and related conditions and symptoms, including celiac disease, associated osteopenia, chronic colitis, Crohn's disease, inflammatory bowel disease and ulcerative colitis); gastric ulcers; hepatic inflammations such as viral and other types of hepatitis, cholesterol gallstones and hepatic fibrosis, HIV infection (and associated conditions and symptoms, including degenerative responses, neurodegenerative responses, and HIV associated Hodgkin's Disease), Kawasaki's Syndrome (and associated diseases and conditions, including mucocutaneous lymph node syndrome, cervical lymphadenopathy, coronary artery lesions, edema, fever, increased leukocytes, mild anemia, skin peeling, rash, conjunctiva redness, thrombocytosis; multiple sclerosis, nephropathies (and associated diseases and conditions, including diabetic nephropathy, endstage renal disease, acute and chronic glomerulonephritis, acute and chronic interstitial nephritis, lupus nephritis, Goodpasture's syndrome, hemodialysis survival and renal ischemic reperfusion injury), neurodegenerative diseases (and associated diseases and conditions, including acute neurodegeneration, induction of IL-1 in aging and neurodegenerative disease, IL-1 induced plasticity of hypothalamic neurons and chronic stress hyperresponsiveness), ophtlialmopathies (and associated diseases and conditions, including diabetic retinopathy, Graves' opthalmopathy, and uveitis, osteoporosis (and associated diseases and conditions, including alveolar, femoral, radial, vertebral or wrist bone loss or fracture incidence, postmenopausal bone loss, mass, fracture incidence or rate of bone loss), otitis media (adult or pediatric), pancreatitis or pancreatic acinitis, periodontal disease (and associated diseases and conditions, including adult, early onset and diabetic); pulmonary diseases, including chronic lung disease, chronic sinusitis, hyaline membrane disease, hypoxia and pulmonary disease in SIDS; restenosis of coronary or other vascular grafts; rheumatism including rheumatoid arthritis, rheumatic Aschoff bodies, rheumatic diseases and rheumatic myocarditis; thyroiditis including chronic lymphocytic thyroiditis; urinary tract infections including chronic prostatitis, chronic pelvic pain syndrome and urolithiasis. Immunological disorders, including autoimmune diseases, such as alopecia aerata, autoimmune myocarditis, Graves' disease, Graves opthalmopathy, lichen sclerosis, multiple sclerosis, psoriasis, systemic lupus erythematosus, systemic sclerosis, thyroid diseases (e.g. goiter and struma lymphomatosa (Hashimoto's thyroiditis, lymphadenoid goiter), sleep disorders and chronic fatigue syndrome and obesity (non-diabetic or associated with diabetes). Resistance to infectious diseases, such as Leishmaniasis, Leprosy, Lyme Disease, Lyme Carditis, malaria, cerebral malaria, meningitis, tubulointerstitial nephritis associated with malaria), which are caused by bacteria, viruses (e.g. cytomegalovirus, encephalitis, Epstein-Barr Virus, Human Immunodeficiency Virus, Influenza Virus) or protozoans (e.g., Plasmodium falciparum, trypanosomes). Response to trauma, including cerebral trauma (including strokes and ischemias, encephalitis, encephalopathies, epilepsy, perinatal brain injury, prolonged febrile seizures, SIDS and subarachnoid hemorrhage), low birth weight (e.g. cerebral palsy), lung injury (acute hemorrhagic lung injury, Goodpasture's syndrome, acute ischemic reperfusion), myocardial dysfunction, caused by occupational and environmental pollutants (e.g. susceptibility to toxic oil syndrome silicosis), radiation trauma, and efficiency of wound healing responses (e.g. burn or thermal wounds, chronic wounds, surgical wounds and spinal cord injuries). Hormonal regulation including fertility/fecundity, likelihood of a pregnancy, incidence of preterm labor, prenatal and neonatal complications including preterm low birth weight, cerebral palsy, septicemia, hypothyroidism, oxygen dependence, cranial abnormality, early onset menopause. A subject's response to transplant (rejection or acceptance), acute phase response (e.g. febrile response), general inflammatory response, acute respiratory distress response, acute systemic inflammatory response, wound healing, adhesion, immunoinflammatory response, neuroendocrine response, fever development and resistance, acute-phase response, stress response, disease susceptibility, repetitive motion stress, tennis elbow, and pain management and response.

Mesenchymal Stem Cells

The MSC of the present invention may be obtained from bone marrow, peripheral blood, skin, hair root, muscle or fat tissue, uterine endometrium, blood, umbilical cord tissue or blood and primary cultures of various tissues.

Preferably, the MSC are isolated from bone marrow, although any source may be used for obtaining MSC for the present invention. By way of example, the bone marrow aspirate may be isolated, washed, and resuspended in media and placed into sterile culture in vitro. Initially, the isolated cells may be plated with serum in the media. The MSC adhere to the culture dish while essentially all other cells are nonadherent and are removed by rinsing (Friedenstein, Exp. Hematol. 4:267-74, 1976). MSC will grow and expand in culture, yielding a well-defined population of pluripotent stem cells. MSC may be further depleted of CD 45 positive cells, by FACS, in order to remove residual macrophages or other hematopoietic cell lineages prior to further expansion, production of MSC CM, or MSC administration to the patient. Preferably, the MSC of the present invention are CD34, CD45 negative, more preferably the MSC are SH2, SH4, CD29, CD44, CD71, CD90, CD106, CD120a positive and CD124, CD14, CD34, CD45 negative. In addition to adherence separation, MSC may be isolated by any technique known to one of skill in the art, including but not limited to, density gradient fractionation, immunoselection, leukapheresis and the like.

The MSC may also be tested morphologically and functionally to show that the isolated stem cells are MSC. For example, a portion of the cells may be cultured in differentiation media to differentiate the MSC into osteocytes and adipocytes as described by Pittenger et al., Science 284: 143-147, 1999. The remaining MSC may be further expanded in culture for administration to the patient, for generation of conditioned media or for cryopreservation for later use.

MSC may be derived from the patient or, under defined circumstances, from a compatible but allogeneic donor. Donor stem cells may be used from a donor having similar compatibility as defined for the organ to be transplanted, including HLA compatibility, known to one skilled in the art. Since MSC can be expanded in vitro, multiple administrations of MSC are possible to further augment the therapeutic effect of the MSC. Use of autologous stem cells eliminates concerns regarding immune tolerance.

The MSC of the present invention may be genetically modified prior to administration to the patient or prior to generation of MSC CM. The MSC may be genetically modified using genes whose products are known to support cellular survival, stimulate cell migration and proliferation, to exert anti-inflammatory actions and to improve intrarenal hemodynamics. Expression of the genes delivered to the MSC may be placed under the control of various promoters, including, but not limited to drug-sensitive promoters that allow both controlled activation and inactivation of these genes. Cloning of the expression vectors for genetically modifying the MSC is performed using materials and methods known to one of skill in the art. Genetic modification of the MSC may be accomplished using methods known to one of skill in the art, including lipofection, calcium phosphate precipitation, infection, including viral vectors, electroporation, and the like.

Endothelial Cells (EC) derived from the MSC described herein by predifferentiation in vitro may also be used in the present invention. The EC may be used for delivery to the patient as described for the MSC or in combination with MSC or MSC CM and combinations thereof. Preparation of the EC for administration is described below.

The practice of the present invention will employ, unless otherwise indicated, conventional methods of virology, microbiology, molecular biology and recombinant DNA techniques within the skill of the art. Such techniques are explained fully in the literature. See, e.g., Sambrook, et al. Molecular Cloning: A Laboratory Manual (Current Edition); DNA Cloning: A Practical Approach, vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, vol. I & II (P. Tijssen, ed.); Fundamental Virology, 2nd Edition, vol. I & II (B. N. Fields and D. M. Knipe, eds.)

MSC Conditioned Media

MSC Conditioned Media (MSC CM) may be obtained by culturing the MSC described above for a time sufficient to condition the media. By way of non-limiting example, the MSC CM may be obtained as follows. MSC may be obtained as described above and the cells plated in culture. MSC that have been depleted of other cells types, for example by adherence plating and removal of CD 45 positive cells by FACS sorting, may be grown to substantially confluent cultures that are essentially contact inhibited. As the cultures are expanding, the MSC may be grown in media containing serum. The MSC may also be grown with autologous serum from the MSC or MSC CM recipient. Once the cultures have expanded to high subconfluence, i.e. about 3−5×10⁶ MSC/T-75 flask, serum may be removed from the media if serum free MSC CM is desired.

The MSC may be grown in normal oxygen conditions, i.e. room air+5% CO₂ (pO₂ approximately 21%). Alternatively and preferably, the MSC may be grown under hypoxic conditions (PO₂≦5%). The media is incubated in the presence of the MSC for a time sufficient to add at least one component to the media that was not present prior to addition of the media to the MSC culture. Preferably, the media is conditioned for one to three days, more preferably for two days. The MSC CM may be collected and filtered though a small pore filter, such as a 0.22 μM filter to sterilize the MSC CM and to remove any particulates. The MSC CM may be administered to the patient or the MSC CM may be frozen, preferably at −120° C., and stored for later administration. The MSC CM may also be concentrated, for example by centrifugation, dialysis, filtration, lyophilization, and the like.

Presence of at least one component added to the media by the MSC may be confirmed using a biological assay, ELISA, or a separation analysis, such as HPLC. For example, the MSC CM may be tested in vitro using proximal renal tubular cells that have been injured, i.e. by scraping, ATP deprivation or both. MSC CM may be added to the cells, using boiled MSC CM or serum free media alone as a control, to evaluate the cells for stimulation of growth, proliferation, and/or survival. The MSC CM may also be tested in vivo. As described above for the MSC, MSC CM may be administered in single, multiple or continuous administrations or combinations thereof. The source of the MSC for generating the CM of the present invention may not require the same level of compatibility as the MSC to be directly implanted into the patient. The generation and use of the MSC CM is described in more detail in the examples provided below.

Administration of a Therapeutically Effective Amount

In certain embodiments, a therapeutically effective amount of MSC is delivered to the patient. In other embodiments, a therapeutically effective amount of MSC CM or EC are administered to the patient. Therapeutically effective amounts of MSC, EC, and MSC CM in any combination thereof may also be administered. An effective amount for treatment will be determined by the body weight of the patient receiving treatment, and may be further modified, for example, based on the severity of the condition, the phase of condition in which therapy is initiated, for example early or advanced, and the simultaneous presence or absence of multiple conditions. The therapeutic amount may also be determined based on the method of delivery to the patient. The therapeutic amount may be one or more administrations of the therapy. Administration of the therapeutic amount of MSC CM may be via continuous infusion, for example, but not limited to a period of 24 hours. Preferably, about 0.01 to about 0.2 ml/100 g body weight MSC CM may be administered in a therapeutic dose, more preferably about 0.04 to about 0.10 ml/100 g body weight MSC CM may be delivered in a therapeutic dose, although other does are possible. Preferably, about 0.01 to about 5×10⁶ cells per kilogram of recipient body weight MSC or EC will be administered in a therapeutic dose, more preferably about 0.02 to about 1×10⁶ cells per kilogram of recipient body weight will be administered in a therapeutic dose. The number of cells used will depend on the weight and condition of the recipient, the number of or frequency of administrations, and other variables known to those of skill in the art. For example, a therapeutic dose may be one or more administrations of the therapy. A subsequent therapeutic dose may include a therapeutic dose of MSC, EC, or MSC CM, or combinations thereof. The therapeutic amount of MSC or MSC CM may be administered to the patient prior to an event inducing the need for treatment, for example, prior to surgery, treatment with chemotherapy, and the like.

Preferably, MSC, EC, and MSC CM may be administered to the patient by injection or instillation intravenously (i.e., large central vein such vena cava) or intra-arterially (i.e., via femoral artery into supra-renal aorta). Any delivery method, commonly known in the art, may be used for delivery of the MSC, EC, and the MSC CM.

Since MSC and EC may be expanded in vitro and MSC CM may be collected and stored, multiple administrations of MSC, EC, and MSC CM are possible to further augment the therapeutic effect of the MSC, EC, and MSC CM. Exemplary patient populations that may benefit from administration of MSC, EC, and MSC CM include, but are not limited to, patients with treatment-resistant (hemodialysis, parenteral nutrition, antibiotics, ICU care) forms of ARF alone or in the setting of MOF or multi-organ dysfunction, patients at highest risk for or who are about to develop the most severe form of treatment-resistant ARF, trauma or surgical patients, scheduled to undergo high risk surgery such as the repair of an aortic aneurysm, patients having infected and non-healing wounds, patients developing MOF post surgery, patients with severe ARF affecting a transplanted kidney and any patients having inflammatory diseases in need of treatment. As discussed above, administration of the therapeutic amount of MSC, EC, or MSC CM may be prior to, during or post development of the condition requiring treatment. Multiple therapeutic amounts may be given to the patients. MSC, EC, and MSC CM therapies may be used for conditions involving all injured organs, including the kidney, lungs, liver, heart, etc.

Assessment of the outcome of the administration of the therapeutically effective amount of MSC, EC, and MSC CM may be assessed by techniques commonly known to one of skill in the art and are not limited to the examples given herein. For example, the treatment of the kidney may be monitored by determination of serum creatinine, BUN, electrolyte levels, measurement of creatinine clearance, urine output, and histology. In experimental models, liver and lung improvement by administration of a therapeutically effective amount may be evaluated by measuring the water content and infiltrating cells in the lungs, and the liver may be evaluated histologically. Biopsy samples of tissues and liver enzymes may also be measured in the patients and experimental models.

Modulation of Growth Factors

MSC, EC, or MSC CM may also be given to a patient in need thereof to modulate expression of growth factors and cytokines in an injured organ. For example, MSC, EC, or MSC CM may be administered to increase expression levels of growth factors and anti-inflammatory cytokines. MSC, EC, or MSC CM may also be administered to decrease expression levels of growth factors and pro-inflammatory cytokines.

Modulation of growth factor expression levels may be measured, for example in the plasma or by measuring the level of expression of the growth factors in the tissues or blood cells. Detection of growth factors in the plasma may be measured using a commercially available ELISA kit such as sold by R & D Systems, Inc. (Minneapolis, Minn.). The expression levels of growth factors in the tissues and blood cells may be determined, for example, by microarray analysis or real time PCR as in the example shown below. Any assay method for measuring modulation of the growth factors known to one skilled in the art may be used.

### EXAMPLES

The invention will now be illustrated by the following non-limiting examples.

### Example 1

### Isolation of MSC

MSC were harvested under anesthesia from femurs of normal adult rats (male or female, Sprague-Dawley or Fisher 344 strain) by flushing the femurs with sterile PBS using a syringe with a 25 gauge needle. The isolated cell aspirates were spun to pellet the cellular content of the aspirate. The pellet was resuspended in culture media (MEM or DMEM/F12 with 10-20% Fetal Calf Serum, Sigma-Aldrich, St. Louis, Mo.), optionally filtered through a 70 μm mesh (Becton & Dickinson, San Jose, Calif.), and plated in 75 cm² primary culture flasks with culture media. Non-adherent cells were removed after 72 hours in culture by repeated rinsing with culture media. Adherent cells were passed at low density into new flasks and expanded to about 3-5×10⁶ MSC/flask. Cells were spindle shaped in appearance. MSC phenotype was confirmed by differentiation into osteocytes and adipocytes with specific differentiation media (Pittenger et al., Science 284: 143-147, 1999). Optionally, the adherent cells were further purified by FACS to eliminate any CD 34 and CD 45 positive cells. MSC were used for administration to the recipient, generation of EC or MSC CM or cyropreserved for later use.

### Example 2

### MSC CM Preparation

MSC isolated as described in Example 1 were used to generate MSC CM. MSC were expanded to high subconfluence, i.e. about 3-5×10⁶ MSC/T-75 flask and the fetal calf serum was removed from the cultures by repeated washing of the MSC with serum free medium. Conditioning of serum free media was then accomplished by culturing MSC under room air (pO_{2˜21}%) or under hypoxic conditions (pO₂≦5%) for 1-3 days. The cell free supernatant (MSC CM) was collected, filtered through a 0.22 μM filter and frozen under sterile conditions at −120° C. Prior to testing in vitro or administration to rats with ARF, the MSC CM was thawed and aliquots were either used without further manipulation or boiled for 20 min at 100° C. prior to administration as a control. The absolute protein concentration in the serum free MSC CM was at the lower detection limit of the biuret protein assay.

### Example 3

### MSC Administration

Ischemia/reperfusion-type of ARF (“ischemic ARF”) was induced in anesthetized rats by timed clamping of both renal pedicles, thereby interrupting the blood supply to the kidneys causing an “ischemic” insult resulting in acute loss of kidney function, i.e., ARF. A model of severe ARF was established using 45 minutes of bilateral renal ischemia. The 45 minute bilateral renal ischemic treatment resulted in a mortality of 50% at 72 hrs post reflow and a glomerular filtration rate of <5% of normal. Histological examination of the severe ARF model shows wide spread tubular necrosis and severe vascular congestion in the corticomedullary junction. A moderate ARF model was established using 35 minutes of bilateral renal ischemia. The moderate ARF model exhibits a serum creatinine level of about 1.5 mg/dL and a mortality of <10%. These models of ARF very closely resemble the most common and most serious form of ARF in patients with shock, sepsis, trauma, after vascular surgery, etc.

MSC were infused intravenously jugular, femoral or tail vein) or intra-arterially (into aorta via carotid or femoral artery) immediately or 24 hrs after induction of ARF. The total number of cells administered in all studies was about 1×10⁵ to 1.5×10⁶ cells/animal. Identical numbers of fibroblasts and identical volumes of serum free media were used as controls.

Renal function in the experimental model was monitored, as in patients, by determination of blood creatinine and BUN levels, measurement of creatinine clearance and urine output. Overall outcome was assessed by determination of weight loss, hemodynamics, and survival. After sacrifice of control and MSC-treated animals with ARF, kidneys were examined for the degree of histological injury (cell apoptosis, necrosis, vascular congestion and injury, inflammatory cell infiltrates) and repair (mitogenesis, redifferentiation of cells, decongestion, etc.).

Histology and injury scores were assessed as follows. Coronal sections of fixed kidneys were stained with H & E and the degree of tubular injury was scored in random cortical fields using a reticule grid with 25 squares with a 20× objective (Chatterjee et al., Calpain inhibitor-1 reduces renal ischemia/reperfusion injury in the rat, Kidney Int. 59: 2073-2083, 2001). One hundred intersections between tubular profiles and the grid were examined for each kidney. Leukocyte infiltration per mm was scored as reported in Togel et al, Hematopoietic stem cell mobilization-associated granulocytosis severely worsens acute renal failure. J. Am. Soc. Nephrol. 15: 1261-1267, 2004.

Immunohistochemistry was performed as follows. Paraffin sections of kidneys were deparaffinized with xylene and rehydrated in an alcohol series and water. After incubation with peroxidase blocking reagent, slides were labeled with a monoclonal mouse anti-rat PC-10 antibody in a ready to use formulation for PCNA staining (DAKO, Carpinteria, Calif.). Scoring for PCNA positive cells, a marker of mitogenesis, was carried out by counting the number of positive nuclei in four randomly chosen sections of kidney cortex and outer medulla with a 20× magnification. Data from all fields and all kidneys were pooled to obtain PCNA scores. Apoptotic scores were obtained with the TUNEL assay using the In Situ Cell Death Detection Kit (Roche, Mannheim, Germany). Kidney sections were deparaffinized, rehydrated and digested with proteinase K and labeled with TUNEL reaction mixture for 60 minutes at 37° C. Sections were screened for positive nuclei under a fluorescence microscope and 10 random sections in the cortex and outer medulla were counted for every kidney under 40× magnification. Data from all fields and all kidneys were pooled to obtain apoptotic scores.

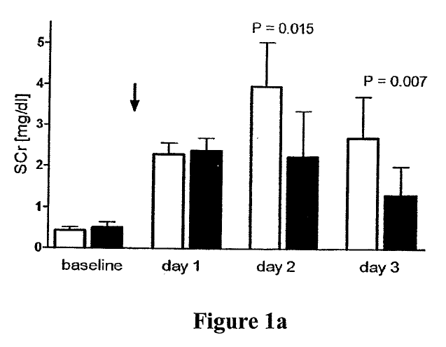

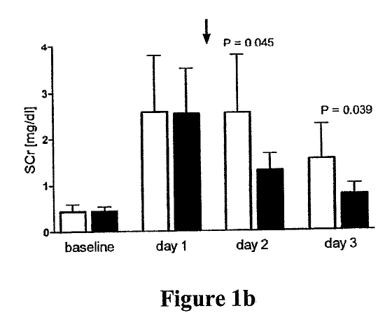

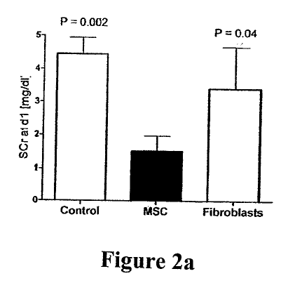

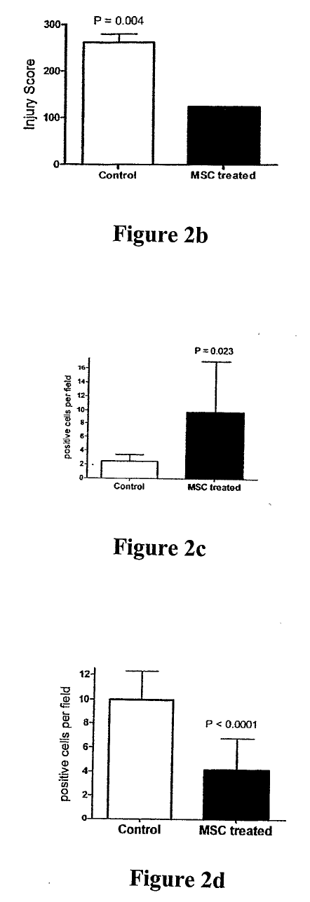

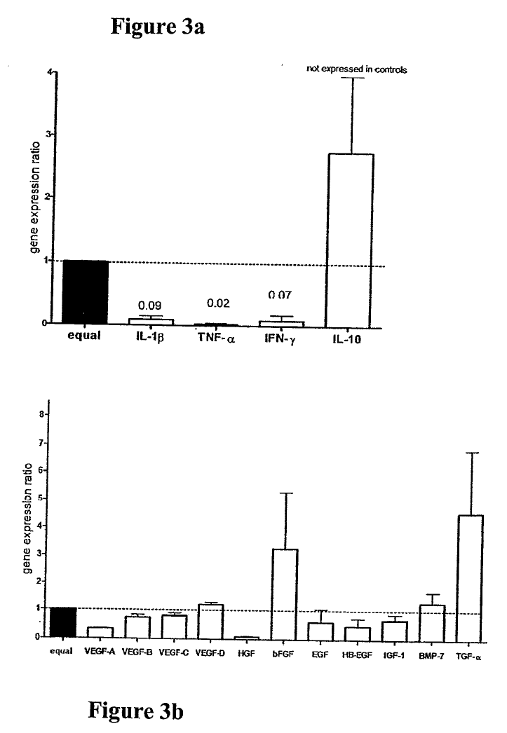

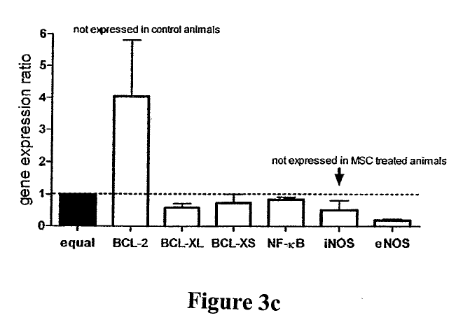

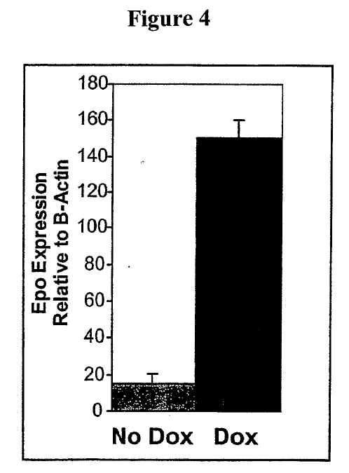

As shown in FIGS. 1a and 1b, administration of MSC directly (1a) or 24 hours (1b) after reflow in rats having moderate ARF significantly improved renal function as determined by serum creatinine. Rats having severe ARF (4.5±0.5 mg/dL serum creatinine at 24 hours in control animals) showed similar improvement in animals given MSC directly after reflow. Serum creatinine levels in treated animals was 2.1±0.5 mg/dL at 24 hours after MSC injection (P=0.002) compared to controls (FIG. 2a). Rats having severe ARF were also assessed for scoring of renal injury, leukocyte infiltration, PCNA and TUNEL staining. MSC treated rats had significantly better injury score (P=0.004) (FIG. 2b), PCNA staining index compared to control animals (P=0.023) (FIG. 2c) and reduced numbers of apoptotic cells in the cortex (P<0.0001) (FIG. 2d). Leukocyte infiltration scores for treated and control animals were similar.

### Example 4

### In Vitro Treatment with MSC CM

MSC CM was prepared as described above in Example 2. In vitro treatment using MSC CM added to injured tubular cells was tested as follows. Normal Rat Kidney Cells (“NRK”, obtained from ATTC, Rockville, Md.), a proximal tubular phenotype, were grown in culture in DMEM with 10% FCS (Hyclone, Logan, Utah), pH 7.40, 37° C., 5% CO₂ and with room air to confluence in 10 cm Petri dishes. Once the NRK were confluent, the cells were washed 3 times with serum free DMEM and the cell media were replaced by serum free DMEM. The NRK were ATP depleted about 75-90% by incubation with 0.1 mM Antimycin (×30 min) and 1 mM 2-Deoxyglucose (×30 min). The NRK were then scrape-wounded with a sterile scalpel (several parallel scrape wounds were generated in the tubular cell monolayer). The degree of ATP depletion was determined in parallel studies using a Luciferase kit (Sigma, St. Louis, Mo.).

The injured cell cultures were carefully rinsed free of Antimycin and 2-Deoxyglucose, and serum free DMEM with MSC CM (room air culture or hypoxic culture) and control media were added to the cultures. The volumes were kept constant and the following additions were made:
MSC CM: 0.2, 0.5, 1.0 ml, total media volume 5 ml.
Boiled MSC CM (20 min, ˜100° C.): 0.2, 0.5, 1.0 ml, total media volume 5 ml.
Hypoxic MSC CM: 0.2, 0.5, 1.0 ml, total media volume 5 ml.
Fetal Calf Serum: 10%, positive control, total media volume 5 ml.
Serum Free Medium: 0.2, 0.5, 1.0 ml, negative control, total media volume 5 ml.

Injured NRK (were assayed at 24 and 48 hours post treatment. There were at least 4-6 independent experiments in each group. As shown in Table 1, motogenesis, mitogenesis, and the degree of apoptosis were measured. Motogenesis measures the amount of cell migration into wounded areas for wound repair. Motogenesis was tested at 0.2 ml MSC CM which was found to be a submitogenic dose.

Mitogenesis measures the amount of cell proliferation and survival and was measured using a MTT assay at 24 and 48 hours. In the MTT assay, the yellow tetrazolium MTT (3-(4,5-dimethylthiazolyl-2)-2,5-diphenyltetrazolium bromide) is reduced by metabolically active cells, in part by the action of dehydrogenase enzymes, to generate reducing equivalents such as NADH and NADPH. The resulting intracellular purple formazan can be solubilized and quantified by spectrophotometric means.

Apoptosis was measured using the TUNEL assay (described above) Annexin-V and PI by FACS analysis using commercially available antibodies; and cytomorphology by immunocytochemistry.

The results are shown in Table 1.

TABLE 1

| | Motogenesis | | Mitogenesis | | Anti-Apoptotic | |
|---|---|---|---|---|---|---|
| | vs. SFM [%] | | vs. SFM [%] | | vs. SFM [%] | |
| Experimental | (0.2 ml CM) | | (1.0 ml CM) | | (1.0 ml CM) | |
| Groups | 24 hrs | 48 hrs | 24 hrs | 48 hrs | 24 hrs | 48 hrs |
| 1) MSC CM | 300* ± 45 | 590* ± 76 | 200* ± 36 | 340* ± 76 | 624* ± 45 | 560* ± 51 |
| 2) Boiled | 96 ± 6 | 101 ± 8 | 99 ± 11 | 103 ± 7 | 97 ± 9 | 99 ± 10 |
| MSC CM | | | | | | |
| 3) Hypoxic | 412* ± 27 | 790* ± 42 | 278* ± 22 | 440* ± 47 | 798* ± 49 | 740* ± 55 |
| MSC CM | | | | | | |
| 4) FCS (10%) | 215** ± 29 | 336** ± 31 | 151** ± 17 | 203** ± 22 | 162** ± 13 | 179** ± 15 |
| positive | | | | | | |
| Control | | | | | | |
| 5) SFM | 100 | 100 | 100 | 100 | 100 | 100 |
| negative | | | | | | |
| Control | | | | | | |

Data in Table 1 are expressed as mean [in %] ± SD of group 5) SFM (=100%), negative control.
*at least p < 0.05, comparing group 1) data to group 2-5 data.
**at least p < 0.05, comparing group 4) to data from groups 2) and 5), and comparing group 4) to group 1).

As shown in Table 1, the MSC CM stimulates significantly motogenesis and mitogenesis and inhibits cell death by apoptosis when compared to the control media in injured cells in culture. The MSC CM affect on motogenesis and mitogenesis and apoptosis is also dose dependent (not shown). MSC CM generated at hypoxic conditions was significantly more potent than MSC CM conditioned at room air. MSC CM harvested at 48 hrs was marginally more potent than that collected at 24 hrs.

### Example 5

### MSC CM Administration In Vivo

MSC CM was prepared as described above in Example 2. MSC CM was injected either in single doses or in a continuous infusion protocol as described below.

For the single doses of MSC CM injected, groups of rats (adult, male SD rats, n=6-8 each) with ischemia/reperfusion induced ARF (I/R ARF) were injected (s.c. or i.v. via tail vein) with 0.2 mL of MSC CM (0.07 mL/100 g) (boiled or unboiled from media conditioned under room air or hypoxic pO₂ or DMEM (SFM) as control) immediately after reflow (following removal of renal pedicle clamps after 40 min of ischemia) and again at 24 hours and 48 hrs. Outcome was assessed during 3 days of observation by recording survival, weights, measurement of renal function (serum creatinine, BUN, renal blood flow) and histological scoring of kidney injury.

The results of the in vivo treatment using single doses of MSC CM are shown in Table 2.

TABLE 2
Single Injections of 0.2 ml MSC CM or SFM post reflow, daily × 3

| | | | | | | | Kidney Injury Score |
|---|---|---|---|---|---|---|---|
| | Serum Cr | | | BUN | | | [1-300]/ |
| Experimental | [mg/dL] | | | [mg/dL] | | | Mortality [%] |
| Groups | d 0 | d 1 | d 3 | d 0 | d 1 | d 3 | d 3 |
| A1) MSC CM | 0.5 ± 0.1 | 1.9* ± 0.2 | 1.6* ± 0.2 | 11 ± 3 | 58* ± 7 | 48* ± 8 | 74* ± 11/0%* |
| A2) Boiled | 0.4 ± 0.1 | 3.4 ± 0.5 | 2.9 ± 0.5 | 13 ± 2 | 125 ± 13 | 114 ± 12 | 225 ± 37/25% |
| MSC CM | | | | | | | |
| A3) Hypoxic | 0.5 ± 0.1 | 1.5* ± 0.3 | 1.1* ± 0.3 | 10 ± 2 | 42* ± 6 | 43* ± 7 | 61* ± 10/0%* |
| MSC CM | | | | | | | |
| A4) SFM | 0.5 ± 0.1 | 3.5 ± 0.6 | 3.0 ± 0.2 | 12 ± 3 | 131 ± 14 | 126 ± 13 | 231 ± 40/20% |
| negative | | | | | | | |
| Control | | | | | | | |

Data in Table 2 are expressed as mean ± SD.
*at least p < 0.05, comparing group A1) data to group A2 & A4) data, and comparing group A1) to group A3) creatinine and BUN data. There were no significant differences between group A2) and A4) data.

As shown in Table 2, neither boiled MSC CM (room air or hypoxic pO₂) nor SFM improved outcomes. The rise in serum creatinine and BUN was significantly less when animals were treated with unboiled MSC CM at 24, 48 and 72 hrs post reflow. In addition, renal blood flow and injury scores of the kidney of rats treated with unboiled MSC CM were significantly better versus those in SFM or boiled MSC CM treated animals. The renoprotective effects of the unboiled MSC CM obtained under hypoxic pO₂ were superior to those obtained with unboiled MSC CM obtained under room air pO₂.

For the continuous infusion protocol, groups of rats (n=4-6 each) were continuously infused into the peritoneum with media (boiled or unboiled MSC CM or SFM as above, 0.2 mL per 24 hrs/300 g), using Alzet miniosmotic pumps. Outcome was assessed during 3 days of observation as above. Outcome was assessed by recording survival, measurement of renal function (serum creatinine, BUN, clearance, renal blood flow) and histological scoring of kidney injury. All variables determined in MSC CM treated rats with ARF were compared to those obtained in boiled MSC CM and SFM treated rats with identical ischemic ARF. All outcome measurements were significantly improved with either type of MSC CM, i.e., room air and hypoxic pO₂. Data for MSC CM generated in room air and hypoxic conditions are shown in Table 3.

TABLE 3
Continuous i.p. Infusion of 0.2 ml MSC CM or SFM/day × 3 days

| | | | | | | | Kidney Injury Score |
|---|---|---|---|---|---|---|---|
| | Serum Cr | | | BUN | | | [1-300]/ |
| Experimental | [mg/dL] | | | [mg/dL] | | | Mortality [%] |
| Groups | d 0 | d 1 | d 3 | d 0 | d 1 | d 3 | d 3 |
| B1) MSC CM | 0.4 ± 0.3 | 2.0* ± 0.4 | 1.5* ± 0.4 | 14 ± 3 | 68* ± 6 | 49* ± 7 | 89* ± 10/0%* |
| B2) Boiled | 0.5 ± 0.2 | 3.5 ± 0.6 | 3.1 ± 0.6 | 13 ± 2 | 136 ± 16 | 123 ± 14 | 239 ± 36/30% |
| MSC CM | | | | | | | |
| B3) Hypoxic | 0.4 ± 0.3 | 1.4* ± 0.3 | 1.1* ± 0.2 | 14 ± 3 | 45* ± 7 | 36* ± 8 | 63* ± 9/0%* |
| MSC CM | | | | | | | |
| B4) SFM | 0.5 ± 0.2 | 3.7 ± 0.6 | 3.2 ± 0.4 | 12 ± 3 | 145 ± 19 | 137 ± 18 | 245 ± 39/25% |
| negative | | | | | | | |
| Control | | | | | | | |

Data in Table 3 are expressed as mean ± SD.
*at least p < 0.05, comparing group B1) data to group B2, B3 & B4) data. There were no significant differences between group B2) and B4) data.

As shown in Table 3, the rise in serum creatinine and BUN was significantly less when animals were treated with unboiled MSC CM at 24, 48 and 72 hrs post reflow. In addition, renal blood flow and injury scores of the kidney were significantly better versus those in SFM or boiled MSC CM treated animals. As shown in Table 3, neither the continuous intraperitoneal infusion of boiled MSC CM nor SFM improved outcomes. The results obtained with single infusions or continuous infusion of MSC CM were not significantly different from each other. The renoprotective effects of the continuously infused unboiled MSC CM obtained under hypoxic pO₂ were also superior to those obtained with unboiled CM obtained under room air pO₂.

### Example 6

### Modulation of Growth Factor Gene Expression with MSC

MSC were obtained and administered to rats having ARF as described above in Examples 1 and 3. Real-time PCR was used to assess the modulation of growth factor and cytokine expression in the kidneys of the rats with and with out MSC administration.

RNA for real time PCR was extracted with the RNeasy kit (Qiagen, Valencia, Calif.), including a DNase digestion step to exclude contaminating DNA. Reverse transcription was performed using M-MLV Reverse Transcriptase (Invitrogen, Carlsbad, Calif.) for 60 min at 42° C.

Real-time PCR with relative quantification of target gene copy numbers in relation to β-actin transcripts was carried out using the following primers:

| | | |
|---|---|---|
| 5′ primer | 3′ primer | SEQ ID |

| | | | |
|---|---|---|---|
| VEGF-A | gcactggaccctggcttt | cggggtactcctggaagatg | [1, 2] |
| VEGF-B | ggaggtggtggtacctctga | gatctgcattcggacttggt | [3, 4] |
| VEGF-C | ccacagtgtcaggcagctaa | actccttgttgggtccacag | [5, 6] |
| VEGF-D | ccggcatccctactcaatta | gcagcgatcttcatcaaaca | [7, 8] |
| HGF | ctcccctgcttcctgtcac | cccttgtttctgatgcacct | [9, 10] |
| EGF | cgtctcagtggtcatggattt | cagaagaacacgggaattgt | [11, 12] |
| BMP-7 | ccaagaggcactgaggatgg | tggtggcgttcatgtaggag | [13, 14] |
| BFGF | cgacccacacgtcaaactac | ccaggcgttcaaagaagaaa | [15, 16] |
| IGF-1 | ggcattgtggatgagtgttg | acgtggcattttctgttcct | [17, 18] |
| HB-EGF | gagaggacggatgagtggtt | ctcaagagttctcgggcttg | [19, 20] |
| IL-1beta | ggacccaagcaccttctttt | agacagcacgaggcattttt | [21, 22] |
| TNFalpha | ctcgagtgacaagcccgtag | ccttgaagagaacctgggagtag | [23, 24] |
| IFNgamma | tctggaggaactggcaaaag | gtgctggatctgtgggttg | [25, 26] |
| IL-10 | cactgctatgttgcctgctc | tgtccagctggtccttcttt | [27, 28] |
| TGF-alpha | cacttcaacaagtgcccaga | agcagtggatcagcacacag | [29, 30] |
| Rat y-chr | cagagatcagcaagcatctgg | tctggttcttggaggactgg | [31, 32] |
| β-actin | agagggaaatcgtgcgtgaca | cactgtgttggcatagaggtc | [33, 34] |

The Smart-Cycler system (Cepheid, Sunnyvale, Calif.) was used to monitor real time PCR amplification using SYBR Green I (Molecular Probes, Eugene, Oreg.), a nonspecific double-stranded DNA intercalating fluorescent dye. All reactions were carried out in a total volume of 25 μL with TaKaRa Ex Taq™ R-PCR Version (TaKaRa Bio Inc, Shiga, Japan). Reaction conditions were: hot start for 120 sec at 95° C., melting at 95° C. for 10 sec, annealing at 63° C. for 12 sec, and amplification at 72° C. for 15 sec. Reading of the fluorescent product was set to be 2° C. below the specific melting peak of the product in order to eliminate reading of nonspecific products and primer dimers and was performed at 85° C. for 6 sec after each cycle for SDF-1. Optimal annealing and melting temperatures were determined for the primers prior to running the samples. Melting temperature analysis for the reaction mix revealed a characteristic melting profile with a single sharp peak at the typical melting temperature for the product. Specificity of the product was determined by a melting curve and gels were run to control for the formation of unspecific bands. Samples were run in duplicate and the average crossing point (CP) value was used for calculations. The CP, which is the cycle at which the amount of amplified gene of interest reached a threshold above background fluorescence, was determined in order to quantitate initial starting copy amount. Relative quantitation of mRNA expression was calculated with the comparative CP method using the following formula:

Ratio=(E_{target})^{ΔCP target(control-sample)}/(E_{ref})^{ΔCPref(control-sample}

(E is the real-time PCR efficiency; CP the crossing point and the difference of a sample versus control.) (Pfaffl et al., Relative expression software tool (REST) for group-wise comparison and statistical analysis of relative expression results in real-time PCR. Nucleic Acids Res. 30: e36, 2002.)

The relative quantitation value of a target gene, normalized to an endogenous control β-actin gene, is expressed as a number, which indicates the relative expression compared to that gene. To avoid the possibility of amplifying contaminating DNA and unspecific amplification, the following precautions were taken: (a) a DNase-digestion step was included in the RNA-extraction protocol, (b) some primers were designed to include an intron sequence inside the cDNA to be amplified, (c) reactions were performed with appropriate negative controls (template-free controls), (d) a uniform amplification of the products was rechecked by analyzing the melting curves of the amplified products (dissociation graphs), (e) gel electrophoresis was performed to confirm both the correct size of the amplification products and the absence of unspecific bands, respectively.

Results of the administration of MSC show changes in the expression of growth factor genes by real time PCR using β-actin expression to normalize expression of each gene as an internal control. At 24 hours post MSC administration, a reduction in the expression of genes encoding the pro-inflammatory cytokines TNF-α, IL-1β, and IFN-γ was shown by real-time PCR (FIG. 3a). MSC treated animals showed an increase in expression of IL-10, an anti-inflammatory cytokine, also shown in FIG. 3a. As shown in FIG. 3b, MSC treated animals had increased expression of bFGF and TGF-α in the kidney and decreased expression of HGF when compared to control kidneys. FIG. 3c shows that Bcl-2 expression was increased while iNOS expression was decreased in MSC treated kidneys compared to control kidneys.

### Example 7

### Genetic Modification of MSC

MSC isolated as described above in Example 1 may be genetically modified prior to administration to a patient or prior to the generation of MSC CM.

Isolated MSC were transduced with retroviral vectors of the Retro Tet-ART system (Rossi et al. Nature Genetics 20: 389-393, 1998) including erythropoietin (EPO). The Phoenix amphotropic cell line was separately transfected with the three retroviral plasmids with FUGENE 6 transfection reagents (Roche, Indianapolis, Ind.). The retroviral plasmids transfected are the following. 1) Expression plasmid HRSp-EPO-IRES-EGFP. In the HRSpuroGUS plasmid of HRSphKGF, GUS was replaced with MnEPO (Beru N, et al., Ann. N.Y. Acad. Sci., 554:29-35, 1989). Expression of mEPO is driven by mCMV, and puromycin is driven by the SV40 promoter. IRES helps the transcription of the 2 genes. 2) TCN-transactivator plasmid. The TCN transactivator binds tet-07 and activates transcription in the presence of Doxycycline (Dox). 3) TCN-transrepressor plasmid. The TCN transrepressor plasmid binds tet-07 and represses transcription in the absence of Dox. Non-regulatable expression of EPO in baboon mesenchymal stem cells has been demonstrated by Bartholomew, A, et al., Hum Gene Ther., 12, 1527-1541, 2001.

The supernatant for transduction of the MSC from the retroviral producer cell line transfected with the three plasmids was harvested 48-hrs post-transfection and passed through a 0.45 μM filter. The target MSC cells were plated at a density of 1×10⁵ cells/ml. For transduction, 8 μg/ml polybrene was added to the retroviral media. The MSC were grown in culture and a portion of the transduced MSC were used to quantitate mEPO gene expression.

First, total RNA was extracted from the transduced MSC with RNAqueous-4PCR kit (Ambion, Austin, Tex.) and 100 ng total RNA was used for one step RT-PCR (Invitrogen, Carlsbad, Calif.).

Next, real time PCR was performed. First strand cDNA was synthesized with 100 ng total RNA using Superscript III RNase H Reverse Transcriptase with Oligo dT (Invitrogen). The standard protocol for the Real Time PCR was used on the Smart Cycler (Cepheid, Sunnyvale, Calif.) to quantitate the mEPO gene expression.

Quantitation of differences between no-Dox vs. Dox treated MSC mEPO transduced samples was performed by One Step RT-PCR and Real Time PCR using the mEPO primers flanking the region in the coding sequence of mEPO cDNA:

| | |
|---|---|
| mEPO torward: | |
| 5′-GGC CAT AGA AGT TTG GCA AG-3′ | (SEQ ID NO: 35) |
| mEPO reverse: | |
| 5′-GTG GTA TCT GGA GGC GAC AT-3′ | (SEQ ID NO: 36) |

As shown in FIG. 4, mEPO gene expression in the transduced MSC mEPO cells is regulatable with Dox treatment.

Using one step RT-PCR, the mEPO primers gave an expected 200 bp PCR product which was verified by sequencing. Dox treatment with 1 μg/ml showed peak EPO gene expression at 24 hrs. The results by real time PCR show ˜4 fold increase in EPO expression in Dox treated samples.

### Example 8

### Modulation of Growth Factor Gene Expression with MSC CM

Similar to the modulation of growth factor gene expression with MSC described in Example 5 above, MSC CM will be used to examine the modulation of growth factor gene expression. MSC CM will be generated and administered as described above in Examples 2 and 4. Real-time PCR will be used to assess the modulation of growth factor and cytokine expression levels in the kidneys of the rats with and without MSC CM administration as described above for the MSC administration. The same primers and conditions will be used as described in Example 5. The results of the administration of MSC CM on the modulation of growth factor gene expression will be assessed as described above in example 5. MSC CM will also be generated from genetically modified MSC, i.e. as described in Example 6, to be used for modulation of growth factor gene expression with MSC CM.

### Example 9

### MSC, EC, or MSC CM and Combinations Thereof

The relative renoprotective and organprotective potency of various treatment protocols will be tested by infusing intravenously jugular, femoral or tail vein) or intra-arterially (into suprarenal aorta via carotid or femoral artery) or intraperitoneally MSC alone, EC alone (preparation described below), MSC CM alone and MSC in combination with EC or MSC CM or EC in combination with MSC CM. Administration will be tested for simultaneous and sequential administrations as well as the timing of administrations, both after onset of the condition and prior to some at risk situations discussed above.

Renal function, histological studies and outcomes in the experimental models will be monitored as detailed above.

### Example 10

### MSC and MSC CM Administration for Multi-Organ Failure

MSC and MSC CM administration will be investigated for boosting the body's ability to cope with the many deleterious consequences of multi-organ failure and for repair and functional recovery of multiple organs. The multi-organ failure model that will be used is the sepsis model in aged rats, in which endotoxin from gram negative bacteria (LPS) is injected and the cecum is perforated, resulting in bacterial peritonitis and all the manifestations of clinical multi-organ failure, including ARF. Improvement in organ function after administration of MSC or MSC CM will be examined. Successful MSC and MSC CM administration is expected to reduce the 100% mortality seen in experimental multi-organ failure, and to significantly enhance wound repair, when applicable. MSC and MSC CM will be administered in therapeutically effective doses separately, in combination, or in serial administrations. EC prepared as described below may also be used for administration in multi-organ failure, in combination with MSC, MSC CM or alone.

### Example 11

### EC Preparation from MSC by In Vitro Differentiation

EC for administration to patients in a therapeutically effective amount will be generated from MSC by differentiation in vitro. The MSC will be plated onto Martrigel® using techniques known to one of skill in the art. (Matrigel is available from BD Biosciences, Franklin Lakes, N.J.) The MSC will be cultured on Martrigel® in media without serum or growth factors for 1-3 days. Alternatively, MSC will be grown on human fibronectin with human VEGF for 7 days or until confluence is reached. Following this protocol, MSC will differentiate into EC phenotype. EC phenotype of cells generated by either method will be verified by showing PECAM-1 (CD 31), von Willebrand Factor, eNOS, and VEGF-Receptor 2 expression, dil-ac-LDL uptake and other suitable markers known to one of skill in the art. EC will then be administered to patients or cryopreserved for future administration as described above for the MSC. In some embodiment, EC will be genetically modified as described above in Example 7. EC will be administered alone or in combination with MSC or MSC CM. Evaluation of the therapeutic effect of the EC will be monitored as described above for the MSC.

### Example 12

### Modulation of Growth Factor Gene Expression with EC

Similar to the modulation of growth factor gene expression with MSC described in Example 5 above, MSC CM will be used to examine the modulation of growth factor gene expression. EC will be generated and administered as described above in Example 11. Real-time PCR will be used to assess the modulation of growth factor and cytokine expression levels in the kidneys of the rats with and without EC administration as described above for the MSC administration. The same primers and conditions will be used as described in Example 5. The results of the administration of EC on the modulation of growth factor gene expression will be assessed as described above in example 5.

Although the invention herein has been described in connection with preferred embodiments thereof, it will be appreciated by those skilled in the art that addition, modifications, substitutions, and deletions not specifically described may be made without departing from the spirit and scope of the invention as defined in the appended claims, and all embodiments that come within the meaning of the claims, either literally or by equivalence, are intended to be embraced therein.

1. A method of treating organ dysfunction, acute renal failure, multi-organ failure, early dysfunction of kidney transplant, graft rejection, chronic renal failure, wounds, and inflammatory disorders, said method comprising delivering a therapeutic amount of a pharmaceutically acceptable media that has been conditioned by exposure to mesenchymal stem cells (MSC) in culture to a patient in need thereof.

2. The method of claim 1 wherein said media is conditioned by exposure to MSC grown in culture conditions where oxygen pressure is less than or equal to 5%.

3. The method of claim 1 wherein said media is conditioned by exposure to MSC grown in culture under room air oxygen conditions.

4. The method of claim 1 wherein said media is conditioned by exposure to said MSC in culture for greater than 24 hours.

5. The method of claim 1 wherein said media has been conditioned by exposure to highly-subconfluent MSC.

6. The method of claim 1 wherein said media is administered systemically.

7. The method of claim 1 wherein said media is administered intra-arterially.

8. The method of claim 1 wherein said media is administered intravenously.

9. The method of claim 1 wherein said media is administered intraperitoneally.

10. The method of claim 1 wherein said media has been conditioned by genetically modified MSC.

11. The method of claim 1 wherein said media is administered by continuous infusion for about 24 hours.

12. The method of claim 1 wherein said media is administered at a dose of 0.07 ml/100 g.

13. The method of claim 1 further comprising delivering a therapeutic amount of MSC to said patient.

14. The method of claim 1 further comprising delivering a therapeutic amount of Endothelial Cells (EC) generated by differentiation of MSC in vitro to said patient.

15. A composition comprising a pharmaceutically acceptable media that has been conditioned by exposure to MSC in culture.

16. The composition of claim 15 wherein said media is serum free.

17. The composition of claim 15 wherein said media is conditioned by exposure to MSC in culture where oxygen pressure is less than or equal to 5%.

18. The composition of claim 15 wherein said media is conditioned by exposure to MSC in culture under room air oxygen conditions.

19. The composition of claim 15 wherein said media has been conditioned by exposure to highly-subconfluent MSC.

20. The composition of claim 15 wherein said MSC have been genetically modified.

21. The composition of claim 15 wherein said media is a concentrated media compared to said media obtained by exposure to MSC in culture.

22. The composition of claim 15 wherein said MSC are human cells.

23. The composition of claim 15 wherein said media has been exposed to said MSC for greater than 24 hours.

24. A method of modulating expression of at least one growth factor in an injured organ of a patient, said method comprising administering an effective amount of MSC, EC, or MSC-conditioned media to the patient to modulate expression of said growth factor.

25. The method of claim 24 wherein said expression is modulated in cells in a kidney of said patient.

26. The method of claim 24 wherein said growth factor is selected from GM-CSF, G-CSF, bFGF and combinations thereof.

27. A method of modulating expression of at least one cytokine in an injured organ of a patient, said method comprising administering an effective amount of MSC, EC, or MSC-conditioned media to the patient to modulate expression of said cytokine.

28. The method of claim 27 wherein said inflammatory cytokine is a pro-inflammatory cytokine.

29. The method of claim 28 wherein said pro-inflammatory cytokine is selected from the group consisting of TNF-α, IL-1β, IL-6, IL-8, lipopolysaccharide-binding protein, MCP-1, CD-14, IFN-γ, a chemokine and combinations thereof.

30. The method of claim 27 wherein said inflammatory cytokine is an anti-inflammatory cytokine.

31. The method of claim 30 wherein said anti-inflammatory cytokine is selected from the group consisting of TNF-RI, TNF-RII, IL-IRA, IL-4, IL-10, IL-12, IL-13, TGF-β, and combinations thereof.


SEQUENCE LISTING (36 sequences)

SEQ ID NO: 1 - DNA, 18 bp, Artificial
  synthetic
gcactggaccctggcttt

SEQ ID NO: 2 - DNA, 20 bp, artificial
  synthetic
cggggtactcctggaagatg

SEQ ID NO: 3 - DNA, 20 bp, Artificial
  synthetic
ggaggtggtggtacctctga

SEQ ID NO: 4 - DNA, 20 bp, Artificial
  synthetic
gatctgcattcggacttggt

SEQ ID NO: 5 - DNA, 20 bp, Artificial
  synthetic
ccacagtgtcaggcagctaa

SEQ ID NO: 6 - DNA, 20 bp, Artificial
  synthetic
actccttgttgggtccacag

SEQ ID NO: 7 - DNA, 20 bp, Artificial
  synthetic
ccggcatccctactcaatta

SEQ ID NO: 8 - DNA, 20 bp, Artificial
  synthetic
gcagcgatcttcatcaaaca

SEQ ID NO: 9 - DNA, 19 bp, Artificial
  synthetic
ctcccctgcttcctgtcac

SEQ ID NO: 10 - DNA, 20 bp, Artificial
  synthetic
cccttgtttctgatgcacct

SEQ ID NO: 11 - DNA, 21 bp, Artificial
  synthetic
cgtctcagtggtcatggattt

SEQ ID NO: 12 - DNA, 20 bp, Artificial
  synthetic
cagaagaacacgggaattgt

SEQ ID NO: 13 - DNA, 20 bp, Artificial
  synthetic
ccaagaggcactgaggatgg

SEQ ID NO: 14 - DNA, 20 bp, Artificial
  synthetic
tggtggcgttcatgtaggag

SEQ ID NO: 15 - DNA, 20 bp, Artificial
  synthetic
cgacccacacgtcaaactac

SEQ ID NO: 16 - DNA, 20 bp, Artificial
  synthetic
ccaggcgttcaaagaagaaa

SEQ ID NO: 17 - DNA, 20 bp, Artificial
  synthetic
ggcattgtggatgagtgttg

SEQ ID NO: 18 - DNA, 20 bp, Artificial
  synthetic
acgtggcattttctgttcct

SEQ ID NO: 19 - DNA, 20 bp, Artificial
  synthetic
gagaggacggatgagtggtt

SEQ ID NO: 20 - DNA, 20 bp, Artificial
  synthetic
ctcaagagttctcgggcttg

SEQ ID NO: 21 - DNA, 20 bp, Artificial
  synthetic
ggacccaagcaccttctttt

SEQ ID NO: 22 - DNA, 20 bp, Artificial
  synthetic
agacagcacgaggcattttt

SEQ ID NO: 23 - DNA, 20 bp, Artificial
  synthetic
ctcgagtgacaagcccgtag

SEQ ID NO: 24 - DNA, 23 bp, Artificial
  synthetic
ccttgaagagaacctgggagtag

SEQ ID NO: 25 - DNA, 20 bp, Artificial
  synthetic
tctggaggaactggcaaaag

SEQ ID NO: 26 - DNA, 19 bp, Artificial
  synthetic
gtgctggatctgtgggttg

SEQ ID NO: 27 - DNA, 20 bp, Artificial
  synthetic
cactgctatgttgcctgctc

SEQ ID NO: 28 - DNA, 20 bp, Artificial
  synthetic
tgtccagctggtccttcttt

SEQ ID NO: 29 - DNA, 20 bp, Artificial
  synthetic
cacttcaacaagtgcccaga

SEQ ID NO: 30 - DNA, 20 bp, Artificial
  synthetic
agcagtggatcagcacacag

SEQ ID NO: 31 - DNA, 21 bp, Artificial
  synthetic
cagagatcagcaagcatctgg

SEQ ID NO: 32 - DNA, 20 bp, Artificial
  synthetic
tctggttcttggaggactgg

SEQ ID NO: 33 - DNA, 21 bp, Artificial
  synthetic
agagggaaatcgtgcgtgaca

SEQ ID NO: 34 - DNA, 21 bp, Artificial
  synthetic
cactgtgttggcatagaggtc

SEQ ID NO: 35 - DNA, 20 bp, Artificial
  synthetic
ggccatagaagtttggcaag

SEQ ID NO: 36 - DNA, 20 bp, Artificial
  synthetic
gtggtatctggaggcgacat